(19) United States
(12) Patent Application Publication
(10) Pub. No.: US 2005/0234466 A1
(43) Pub. Date: Oct. 20, 2005

(54) TLS adjustable block

(76) Inventor: Jody Stallings, Palm Harbor, FL (US)

Correspondence Address: CHIEF PATENT COUNSEL;SMITH & NEPHEW, INC., 1450 BROOKS ROAD, MEMPHIS, TN, 38116, US

(21) Appl. No.: 11/096,809

(22) Filed: Mar. 31, 2005

Related U.S. Application Data
(60) Provisional application No. 60/558,208, filed on Mar. 31, 2004.

(51) Int. Cl. A61B 17/58

(52) U.S. Cl. 606/88

(57) ABSTRACT

Adjustable and modular systems, devices and methods for accurately cutting or resecting bones during surgery, particularly in preparation for installing joint implants during arthroplasties, including, but not limited to, preparation of femur or tibia during knee arthroplasties, such as total knee arthroplasty. The embodiments of the present invention provide solutions for adjusting a position of the cutting guides, or structures for guiding or directing the implements for resecting a patient's bone tissue, such as saws. The systems and devices comprise a base, an attachment member for securing the base to a bone, and adjustment members extending from the base to contact surfaces of the bone and allowing adjustment of the position of the base.

### CROSS-REFERENCE TO RELATED APPLICATION

This application claims the benefit of U.S. Provisional Patent Application Ser. No. 60/558,208 entitled “TLS Adjustable Block” filed on Mar. 31, 2004, the entire content of which is incorporated herein.

### FIELD OF THE INVENTION

The present invention relates generally to systems, devices and methods for preparing bones for installing joint implants during joint replacement surgery. More specifically, the present invention relates to adjustable systems for cutting bones during joint replacement surgery, particularly to adjustable surgical cutting blocks for resecting femoral or tibial bones, or both, during total knee replacement surgery, or total knee arthroplasty.

### BACKGROUND

Joint implants, also referred to as joint prostheses, joint prosthetic implants, joint replacements, or prosthetic joints, are long-term surgically implantable devices that are used to partially or totally replace within the musculoskeletal system of a human or an animal diseased or damaged joints, such as, but not limited to, a knee, a hip, a shoulder, an ankle, or an elbow joint. Since their first introduction into clinical practice in the 1960s, joint implants have improved the quality of life of many patients.

Knee arthroplasty is a procedure for replacing components of a knee joint damaged by trauma or disease. During this procedure, a surgeon removes a portion of one or more knee bones forming the knee joint and installs prosthetic components to form the new joint surfaces. In the United States alone, surgeons perform approximately 250,000 total knee arthroplasties (TKAs), or total replacements of a knee joint, annually. It is desirable to improve knee arthroplasty devices, instruments, and techniques to ensure better restoration of knee joint function and shorten the patient's recovery time.

The human knee joint essentially includes four bones. The lower extremity of the femur, or distal femur, attaches by ligaments and a capsule to the proximal tibia. The distal femur contains two rounded oblong eminences, the condyles, separated by an intercondylar notch. A trochlear groove, on the anterior aspect of the distal femur, also lies between the condyles. The tibia and the femur do not interlock but meet at their ends. The femoral condyles rest on the condyles of the proximal tibia. The fibula, the smaller shin bone, attaches just below the tibia and is parallel to it. The patella, or knee cap, is at the front of the knee protecting the joint and providing extra leverage. A patellar surface is a smooth shallow articular depression between the femoral condyles at the front. Cartilage lines the surfaces of the knee bones, cushions them, and minimizes friction. Two C-shaped menisci, or meniscal cartilage, lie between the femur and the tibia, serve as pockets for the condyles, and stabilize the knee. Several ligaments connect the knee bones and cover and stabilize the joint. The knee ligaments include the patellar ligament, the medial and lateral collateral ligaments, and the anterior (ACL) and posterior (PCL) cruciate ligaments. Ligaments and cartilage provide the strength needed to support the weight of the upper body and to absorb the impact of exercise and activity. A bursa, or sack, surrounds the knee joints and contains lubricating fluid.

A healthy knee allows the leg to move freely within its range of motion while supporting the upper body and absorbing the impact of its weight during motion. The knee has generally six degrees of motion during dynamic activities: three rotations (flexion/extension angulations, axial rotation along the long axis of a large tubular bone, also referred to as interior/exterior rotation, and varus/valgus angulations); and three translations (anterior/posterior, medial/lateral, and superior/inferior).

A total knee arthroplasty, or TKA, replaces both the femoral component and the tibial component of the damaged or affected by disease knee with artificial components made of synthetic materials, including, but not limited to, metals, ceramics, plastics, or combinations of them. These prosthetic knee components are attached to the bones, and existing ligaments and muscles are used to stabilize the artificial knee. During TKA, after preparing and anesthetizing the patient, the surgeon makes a long incision along the front of the knee and positions the patella to expose the joint. After exposing the ends of the bones, the surgeon removes the damaged tissue and cuts, or resects, the portions of the tibial and femoral bones to prepare the surfaces for installation of the prosthetic components. After preparation of the bones, the knee is tested with the trial components. Ligament balancing, including any necessary surgical release or contraction of the knee ligaments, is performed to ensure proper selection of the prosthetic components and post-operative functioning of the knee. Both anatomic (bone-derived landmarks) and dynamic or kinematic (ligament and bone interactions during the knee movement) data are usually considered when determining surgical cuts and positioning of the prosthetic components. After ligament balancing and proper selection of the components, the surgeon installs and secures the tibial and femoral components. The patella is resurfaced before or after installation of the tibial and femoral component, and a small plastic piece is often placed on the rear side, where it will cover the new joint. After installation of the knee prosthesis, the knee is closed according to conventional surgical procedures. Post-operative rehabilitation starts shortly after the surgery to restore the knee's function.

Improper positioning and misalignment of the prosthetic knee components commonly cause prosthetic knees to fail, leading to revision surgeries. This failure increases the risks associated with knee replacement, especially because many patients requiring prosthetic knee components are elderly and highly prone to the medical complications resulting from multiple surgeries. Also, having to perform revision surgeries greatly increases the medical costs associated with the restoration of the knee function. In order to prevent premature, excessive, or uneven wear of the artificial knee, the surgeon must implant the prosthetic device so that its multiple components articulate at exact angles. Thus, correctly preparing the bone for installation of the prosthetic components by precisely determining and accurately performing all the required bone cuts is vital to the success of TKR.

The surgeons generally rely heavily on their experience to determine where the bone should be cut. They also use various measuring and indexing devices to determine the location of the cut, and various guiding devices, such as, but not limited to, guides, jigs, blocks and templates, to guide the saw blades to accurately resect the bones. After determining the desired position of the cut, the surgeon usually attaches the guiding device to the bone using appropriate fastening mechanisms, including, but not limited to, pins and screws. Attachment to structures already stabilized relative to the bone, such as intramedullary rods, can also be employed. After stabilizing the guiding device at the bone, the surgeon uses the guiding component of the device to direct the saw blade in the plane of the cut.

To properly prepare femoral surfaces to accept the femoral component of the prosthetic knee, the surgeon needs to accurately determine the position of and perform multiple cuts, including, but not limited to, a transversely directed distal femoral cut, an axially directed anterior femoral cut, an axially directed posterior femoral cut, anterior and posterior chamfer femoral cuts, a trochlear recess cut, or any combination or variation of those. Preparation of the tibia for installation of the tibial component may also involve multiple cuts. Sequentially attaching to the bone and properly positioning a series of cutting guides, each adapted for a specific task, lengthens and complicates the TKR procedure. This problem is particularly pressing in the context of the so-called “minimally invasive surgery” (MIS) techniques.

The term “minimally invasive surgery” generally refers to the surgical techniques that minimize the size of the surgical incision and trauma to tissues. Minimally invasive surgery is generally less intrusive than conventional surgery, thereby shortening both surgical time and recovery time. Minimally invasive TKA techniques are advantageous over conventional TKA techniques by providing, for example, a smaller incision, less soft-tissue exposure, improved collateral ligament balancing, and minimal trauma to the extensor mechanism (see, for example, Bonutti, P. M., et al., Minimal Incision Total Knee Arthroplasty Using the Suspended Leg Technique, Orthopedics, September 2003). To achieve the above goals of MIS, it is necessary to modify the traditional implants and instruments that require long surgical cuts and extensive exposure of the internal knee structures. To make the knee implants and knee arthroplasty instruments, structures, and devices particularly suitable for minimally invasive surgical procedures, it is desirable to decrease their size and the number of components. Cutting systems and devices for MIS are desired that can be installed and adjusted with minimal trauma to the knee's tissues and allow the surgeon to perform the cuts quickly and efficiently without compromising the accuracy of the resection. Also desired are cutting systems and devices that minimize the number of surgical steps required to accurately cut the bones in preparation for installation of the prosthetic knees.

Another recent development in TKA is computer-assisted surgical (CAS) systems that use various imaging and tracking devices and combine the image information with computer algorithms to track the position of the patient's leg, the implant, and the surgical instruments and make highly individualized recommendations on the most optimal surgical cuts and prosthetic component selection and positioning. Several providers have developed and marketed imaging systems based on CT scans and/or MRI data or on digitized points on the anatomy. Other systems align preoperative CT scans, MRIs, or other images with intraoperative patient positions. A preoperative planning system allows the surgeon to select reference points and to determine the final implant position. Intraoperatively, the system calibrates the patient position to that preoperative plan, such as using a “point cloud” technique, and can use a robot to make femoral and tibial preparations. Other systems use position and/or orientation tracking sensors, such as infrared sensors acting stereoscopically or otherwise, to track positions of body parts, surgery-related items such as implements, instrumentation, trial prosthetics, prosthetic components, and virtual constructs or references such as rotational axes which have been calculated and stored based on designation of bone landmarks. Processing capability such as any desired form of computer functionality, whether standalone, networked, or otherwise, takes into account the position and orientation information as to various items in the position sensing field (which may correspond generally or specifically to all or portions or more than all of the surgical field) based on sensed position and orientation of their associated fiducials or based on stored position and/or orientation information. The processing functionality correlates this position and orientation information for each object with stored information regarding the items, such as a computerized fluoroscopic imaged file of a femur or tibia, a wire frame data file for rendering a representation of an instrumentation component, trial prosthesis or actual prosthesis, or a computer generated file relating to a rotational axis or other virtual construct or reference. The processing functionality then displays position and orientation of these objects on a screen or monitor, or otherwise. The surgeon may navigate tools, instrumentation, trial prostheses, actual prostheses and other items relative to bones and other body parts to perform TKAs more accurately, efficiently, and with better alignment and stability.

With the introduction of the computer-assisted surgical systems, adjustable systems for cutting the bone during TKR became particularly desired. Although some providers developed adjustable cutting blocks, their adjustment capabilities were generally limited to setting a parameter, such as the varus/valgus angle, prior to installation of the cutting block The cutting systems capable of being adjusted continuously during surgery were not desirable, because the surgeon was not able to follow the position of the installed cutting block after adjustment. Once the computer-aided systems and processes became available that can provide useful data throughout TKR surgery on predicted or actual position and orientation of body parts, surgically related items, implants, and virtual constructs for use in navigation, assessment, and otherwise performing surgery or other operations, cutting systems became particularly desirable whose position can be continually adjusted after taking into account the feedback from the computer functionality. Additionally, the known adjustable cutting systems are not suitable for minimally invasive surgery, because they are generally too large to be placed in a small incision, too cumbersome to use, and require additional mechanical referencing devices for proper positioning and adjustment.

U.S. patent application Ser. No. 10/989,835 to McGinley et al. filed Nov. 15, 2004 provides one type of solution to these problems. This application is incorporated herein by this reference. However, alternative and complementary systems for guiding bone cuts during TKR may provide additional benefits in minimally invasive surgery, computer-assisted surgery, or both. To this end, alternative and complementary cutting systems or devices are needed that also allow the surgeon to minimize the size of the surgical incision and tissue damage, thereby reducing the surgical repairs and shortening the recovery time. Cutting systems and devices are needed that minimize damage to the bone during installation, that can be positioned and installed at the bone without the encumbrances of mechanical referencing devices, and whose position can be precisely controlled before and after installation so that it is possible to place them accurately in the desired location suggested by a navigation system.

Systems and devices are also desired that are adjustable in multiple angles of rotation and multiple translations, but miniature enough to be useful for minimal invasive surgery, thereby reducing patient visit time and costs, and potential of infection. In general, surgical cutting guides are needed for use in TKA that are easy to use and manufacture, minimize tissue damage, simplify surgical procedures, are robust, can withstand multiple surgeries and required sterilization treatments, are versatile, allow for faster healing with fewer complications, require less post-surgical immobilization, are simple to use so as to require less operator training, and also less costly to produce and operate.

### SUMMARY

The aspects and embodiments of the present invention provide novel systems, devices and methods for accurately cutting or resecting bones during surgery. In a preferred embodiment, the systems, devices, and methods are for resecting bones in preparation for installing joint implants during arthroplasties, including, but not limited to, preparation of the femur or tibia during knee arthroplasties, such as total knee arthroplasty. Certain aspects and embodiments of the present invention provide novel solutions for adjusting the position of a cutting guide or other structure for guiding or directing implements for resecting a patient's bone tissue, such as a saw. The systems and devices according to aspects and embodiments of the present invention are also finely adjustable.

The systems and devices for positioning a cutting guide according to one embodiment of the present invention comprise an attachment member for connecting a base to bone to be resected using the cutting guide, a base adapted to connect to the attachment member in a single connection and adapted to move relative to the attachment member through a plurality of positions in a predetermined range of motion in at least two degrees of rotational freedom and one degree of translational freedom, and a plurality of adjustment members extending from the base to the bone, the adjustment members adapted to be adjusted in order to adjust the orientation of the base relative to the bone in at least two degrees of rotational freedom. The base may include or be attached to a cutting guide. During adjustment, the adjustment members are operably connected to the base that has some freedom to move relative to the patient. Manipulating the adjustment members adjusts the position of the base, thereby adjusting the cutting block and its position relative to the bone.

Compared to conventional adjustable cutting guides and systems, the systems according to aspects and embodiments of the present invention advantageously allow a user to adjust the position of cutting guides relative to a patient throughout the surgical procedure. Many conventional systems fail to provide for adjustment of position of the cutting guides after their initial installation. They have to be adjusted prior to their installation in the surgical field, forcing the user to rely on the preliminary estimates of the cutting guide's position, not necessarily accurate. In contrast, the systems according to the aspects and embodiments of the present invention are initially generally located and installed relative to the patient based on any suitable technique available to the user, followed by precisely adjusting the position of the cutting guide by manipulating the base module. Upon adjustment, the cutting guide may be affixed or otherwise stabilized relative to the bone and is used to direct the cutting implement in bone resection.

The modular structure of the systems and devices according to the aspects and embodiments of the present invention increases their versatility compared to conventional devices. Further improving the system's versatility, the base can be stabilized with respect to the bone either by directly attaching them to the bone, or indirectly, by attaching the base to structures affixed or stabilized with respect to the patient. For example, a base can be attached to pre-installed intramedullary rods or anchor posts, thereby providing and additional opportunity for positioning relative to the patient.

The adjustability of the systems and devices according to aspects and embodiments of the present invention allows their installation in a variety of patients and their use for preparation of bones differing in size and shape in different surgical applications. By incorporating multiple adjustment capabilities, the dimensions and position of the systems and devices according to aspects and embodiments of the present invention are easier and more accurate to adjust than those of conventional devices.

Although suitable for a variety of applications, the modular adjustable systems and devices according to aspects and embodiments of the present invention are particularly advantageous for minimally invasive surgeries, such as minimally invasive knee arthroplasty. The cutting systems and devices according to aspects and embodiments of the present invention are generally smaller than conventional cutting systems and devices, although their size can be adjusted to the needs of a particular surgical procedure. For installation, the systems and devices can be separated into modules. The adjustment structures and mechanisms are advantageously smaller in size and, in certain embodiments, integrate multiple adjustment capabilities, thereby reducing the total number and size of the requisite components. Employing one or more of the foregoing principles minimizes the size of the needed surgical incisions, minimizes tissue damage in general, reduces surgical repairs, and shortens the recovery time.

The modular adjustable systems and devices according to aspects and embodiments of the present invention are also particularly advantageous for computer assisted surgical procedures, such as computer-assisted knee arthroplasty. The position of the cutting systems and devices can be precisely controlled before and after installation. Thus, it is possible to fine-tune their position throughout surgery using navigational feedback.

The capabilities of the cutting systems and devices that allow their use in conjunction with computer-assisted surgery systems further minimize the damage to the patient's tissues and improve the recovery as compared to the conventional systems. In one aspect, this is because the cutting systems and devices can be positioned and installed at the bone without the encumbering mechanical referencing devices. In another aspect, the cutting systems and devices are accurately adjustable in multiple degrees of freedom, thereby allowing for more precise fit and control of the position than conventional devices, thereby achieving more accurate bone cuts and better fit of the joint prosthetic components, reducing the prosthetic's failure rate and the need for subsequent revision surgeries, and improving the patient's restoration of function.

Embodiments of the present invention also provide methods for adjusting a position of a cutting block at a bone during surgery using systems and devices according to the aspects and embodiments of the present invention. One embodiment of the invention includes a method of arthroplasty surgery on a knee comprising positioning the knee in a flexed position, exposing a joint of the knee, attaching a base to the bone through an attachment member, whereby the base is adapted to connect to the attachment member in a single connection and is adapted to move relative to the attachment member through a plurality of positions in a predetermined range of motion in at least two degrees of rotational freedom and one degree of translational freedom, adjusting the position of the base by manipulating a plurality of adjustment members extending from the base to contact surfaces of the bone, whereby the adjustment members are adapted to be adjusted in order to adjust the orientation of the base relative to the bone in at least two degrees of rotational freedom, and cutting the bone with a cutting implement directed by a cutting guide wherein the cutting guide is attached to the base.

The systems and devices according to certain embodiments of the present invention are adjustable in multiple degrees of freedom, including one or more angles of rotation and one or more translations, and are modular, with one or more modules miniature enough for minimally invasive surgery. In general, the systems according to the embodiments provided herein reduce patient visit time and costs and potential of infection. They are easier to use and manufacture, minimize tissue damage, simplify surgical procedures, are robust, can withstand multiple surgeries and required sterilization treatments, are versatile, allow for faster healing with fewer complications, require less post-surgical immobilization, are simple to use so as to require less operator training, and are also less costly to produce and operate.

### BRIEF DESCRIPTION OF THE DRAWINGS

FIG. 1 is an isometric view of an adjustable cutting system attached to an end of a tubular bone according to one embodiment of the invention.

FIG. 2 is a side view in the medial/lateral direction of the adjustable cutting system of FIG. 1 attached to an end of a tubular bone.

FIG. 3 is a front view in the anterior/posterior direction of the adjustable cutting system of FIG. 1 attached to an end of a tubular bone.

FIG. 4 is a top view in the superior/inferior direction of the adjustable cutting system of FIG. 1 attached to an end of a tubular bone.

### DETAILED DESCRIPTION

The advantages of the systems according to certain aspects and embodiments of the present invention are achieved by providing, for example, a system for preparation of a bone of a patient during total knee arthroplasty, such as the systems for resection of distal femur or proximal tibia in preparation of installation of the femoral and the tibial components, respectively, during total knee arthroplasty. However, the application principles and structures illustrated herein by the embodiments of the present invention are not limited to resection of distal femur or distal tibia and are not limited to total knee arthroplasty. Various other uses of devices according to aspects and embodiments of the present invention are envisioned, such as, but not limited to, use in joint arthroplasty, including various knee arthroplasties, and for resection of bone tissue in any surgical procedure where precise and accurate cuts are beneficial.

Systems for positioning a cutting guide according to aspects and embodiments of the present invention can comprise a base, an attachment member for securing the base to a bone, and adjustment members extending from the base to contact surfaces of the bone.

Systems and devices for positioning a cutting guide according to one embodiments of the present invention comprise an attachment member for connecting a base to bone to be resected using the cutting guide, a base adapted to connect to the attachment member in a single connection and adapted to move relative to the attachment member through a plurality of positions in a predetermined range of motion in at least two degrees, or axes, of rotational freedom and one degree of translational freedom, and a plurality of adjustment members extending from the base to the bone, the adjustment members adapted to be adjusted in order to adjust the orientation of the base relative to the bone in at least two degrees of rotational freedom. The base may include or be attached to a cutting guide.

The adjustment members are operably connected to the base that has some freedom to move relative to the patient over a predetermined range of motion. The predetermined range of motion may include a continuously positionable range or a range having only a fixed number of incrementally separated positions, among others. The predetermined range of motion may determined by adjustment members or by the connection between the adjustment members and the base. For example, a series of notches may be used to allow a turning adjustment member to be adjusted in small increments.

Manipulating the adjustment members adjusts the position of the base, thereby adjusting the cutting block and its position relative to the bone. The adjustment members may be adjusted to properly orient the cutting guide, which is used to direct an implement for resecting the bone, for example, a surgical saw.

Adjustment of the cutting guide may involve adjustment one or more degrees of rotational freedom and one or more degrees of translational freedom. In reference to the knee joint, the degrees of rotational freedom are commonly referred to as varus/valgus angle, flexion/extension angle, and the internal/external axial rotation, or rotation around the long axis of a large tubular bone. The degrees of translational freedom are commonly referred to as superior/inferior (height along the long axis of a large tubular bone), medial/lateral, and anterior/posterior. It is to be understood that the adjustment capabilities of the systems provided herein are not limited by the above terms and other notations for denoting degrees of rotational and translational freedom can be used.

The base comprises structures for attaching to the patient. Such structures include, but are not limited to, structures for connecting the base to a bone, such as openings for inserting attachment pins or screws, spikes, or the like. Attaching or affixing the base to the patient can be performed in a variety of ways, including direct attachment to the bone, or by engaging a structure or a surgical device fixed relative to the patient, such as, but not limited to, an anchor post, an extramedullary rod, or an intramedullary rod inserted into a bone.

Generally the base will connect to the attachment member, or structure attaching it to the bone, at a single connection. The base may be adapted to move relative to the attachment member through a plurality of positions in a predetermined range of motion in at least two degrees of rotational freedom and one degree of translational freedom.

The base may further comprise a cutting guide or structures for engaging, attaching to, or otherwise connecting to one or more cutting guides. Cutting guides are also referred to as cutting blocks, jigs, or by other terms. Various cutting guides may be used. Typically, a cutting guide will comprise one or more structures, such as a guiding slot or a guiding plane, for directing a cutting implement.

Aspects and embodiments of the present invention can provide multiple adjustment capabilities to the surgical cutting guides without increasing their size or number of components. A cutting guide according to certain aspects and embodiments of the present invention further comprises structures and devices for attaching the guide to a bone, such as the distal femur or proximal tibia, prior to resection.

In certain aspects and embodiments, adjustment members extend from the base to contact surfaces of the bone. The adjustment members are operably connected to the base. In one embodiment the adjustment members are screws that interact with screw apertures in the base. For example, three screws could pass through holes in the base and extent to contact the surface of the bone. In this embodiment, the rotational and translational position of the base may be adjusted by adjusting one or more of the three screws. In some embodiment a cutting block is connected to the base, so that manipulating the screws, or other adjustment members, to adjust the position of the base, also adjusts the cutting block and its position relative to the bone. A surgeon can make fine adjustments to the position of the cutting block by adjusting the adjustment members.

The change of position of the base and/or cutting block can be translational or rotational or both. The block and base may be connected by one or more structures, including but not limited to, interlocking parts, rail/slot structures, t-slots, clamps, screws, pins, racks, or ball-and-socket joints.

Systems and devices of embodiments of the present invention can also comprise various structures that allow fine adjustment of the position of the base and/or cutting block. These structures may include, among others, the adjustment members between the base and the bone and the attachment between the base and the cutting block. These structures which allow the manipulating of the position of the base and/or cutting block, may include components such as knobs, screws, levers, or the like.

Systems and devices of embodiments of the present invention can be adapted as needed for manipulation and adjustment by a user, such as a surgeon, with or without the input of a computer functionality, an automatic, robotic, or computer-aided navigating or manipulating device, or any combination or variation of the foregoing.

In a particular embodiment of the present invention, the user employs the systems and devices to adjust the position of a cutting guide during knee surgery, such as TKA. Accordingly, the cutting guide is a femoral cutting guide for distal femoral resection or a tibial cutting guide for proximal tibial resection. The cutting guide can be for guiding a saw in one or more cuts. For example, the femoral cutting guide is a guide for performing one or more femoral cuts, including, but not limited to, the cuts of the distal femur, such as, distal, axially directed anterior, axially directed posterior, anterior chamfer, or posterior chamfer cuts, or a combination thereof. Integrating several guiding capabilities in the same guide, or providing the capability to engage several cutting guides to a base, simultaneously or sequentially, advantageously reduces the number of components required for complete preparation of the bone. This reduction, in turn, minimizes the complexity and the size of the cutting system, rendering it particularly suitable for, although not limited to, minimally invasive surgical applications.

The base according to aspects and embodiments of the present invention may have one or more structures for adjusting the position of a cutting guide at a patient's bone, such as a tibial or a femoral bone, in one or more of superior/inferior, medial/lateral, or anterior/posterior translations. The cutting guide base may also have one or more structures for adjusting the position of the cutting guide at a patient's bone, such as a tibial or a femoral bone, in one or more of varus/valgus angle, flexion/extension angle, or axial rotation. A femoral cutting guide base according to one of the embodiments of the present invention comprises one or more structures for adjusting the position of a cutting guide with respect to the femur in at least one of varus/valgus angle, flexion/extension angle, or proximal/distal translation. In one embodiment, three adjustment members are used to adjust the position of a cutting guide in at least two degrees of rotational freedom, varus/valgus angle, flexion/extension. In another embodiment, a connection between the base and the cutting block is used to adjust the resection depth of the cutting guide.

The adjustments devices and techniques of the present invention may be used in combination with other adjustment techniques to enhance functionality. Providing multiple adjustment capabilities is useful in that mechanisms best suited for each adjustment step can be employed. For example, a slidable rail/slot, lever-controlled connection can be used for gross translational adjustment in a degree of freedom, whereas a screw-controlled adjustment members can be employed for fine-tune adjustment. Providing mechanisms for both gross and fine adjustment control in the same system allows for more precise control of the location of the cutting block than that allowed by the conventional cutting blocks. It is also advantageous in computer-assisted surgical applications. For example, during computer-assisted surgery, the user provisionally locates the cutting block using conventional anatomical landmarks, and then fine-tunes the block's position using navigational feedback from the computer functionality.

Systems and devices according to aspects and embodiments of the present invention can include computer functionalities, imaging or navigation functionalities, or other aspects and components or systems for computer-aided surgery, or be integrated or interfaced with such systems. Systems and devices according to aspects and embodiments of the present invention can include aspects and components or systems for minimally invasive surgery, or be integrated or interfaced with such systems.

Methods for adjusting a position of a cutting block at a bone during surgery using systems and devices according to aspects and embodiments of the present invention generally comprise the following elements, not necessarily in the listed order, positioning the knee in a flexed position, exposing a joint of the knee, attaching a base to the bone through an attachment member, whereby the base is adapted to connect to the attachment member in a single connection and is adapted to move relative to the attachment member through a plurality of positions in a predetermined range of motion in at least two degrees of rotational freedom and one degree of translational freedom, adjusting the position of the base by manipulating a plurality of adjustment members extending from the base to contact surfaces of the bone, whereby the adjustment members are adapted to be adjusted in order to adjust the orientation of the base relative to the bone in at least two degrees of rotational freedom; and cutting the bone with a cutting implement directed by a cutting guide wherein the cutting guide is attached to the base.

Methods according to certain aspects and embodiments of the present invention can further comprise the step of adjusting the resection depth of the cutting guide by adjusting a connection between the base and the cutting guide.

The foregoing discloses embodiments of the present invention, and numerous modifications or alterations may be made without departing from the spirit and the scope of the invention.

### Exemplary Cutting System

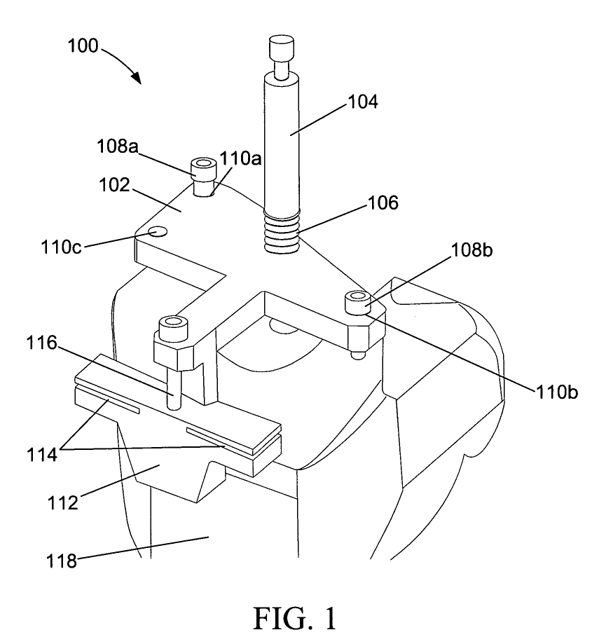

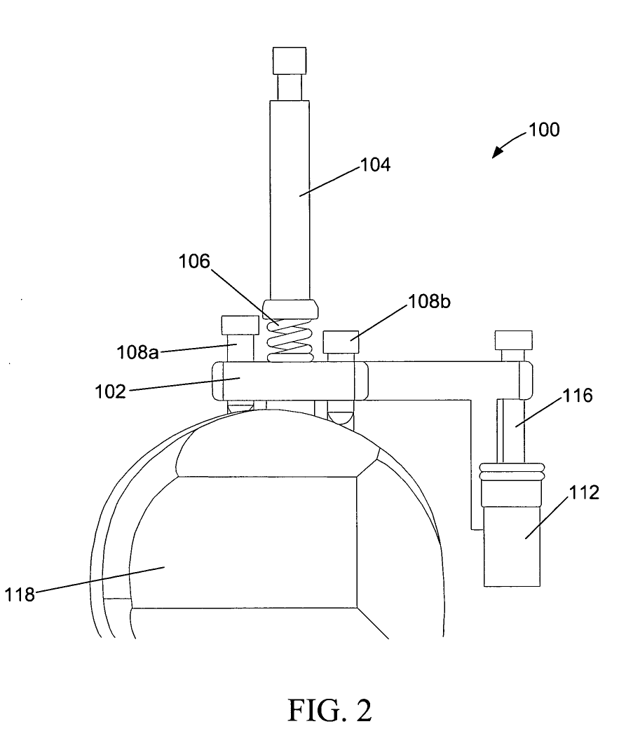

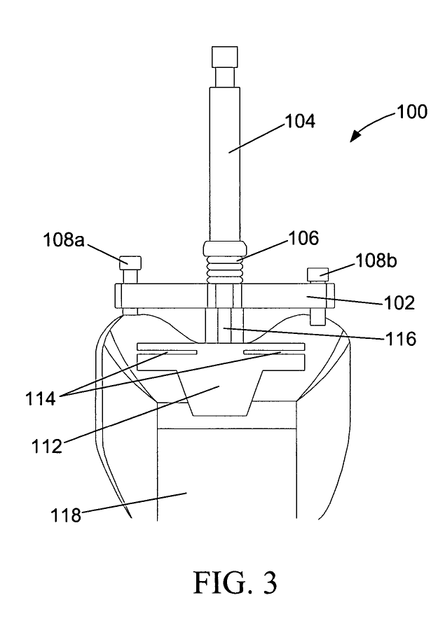

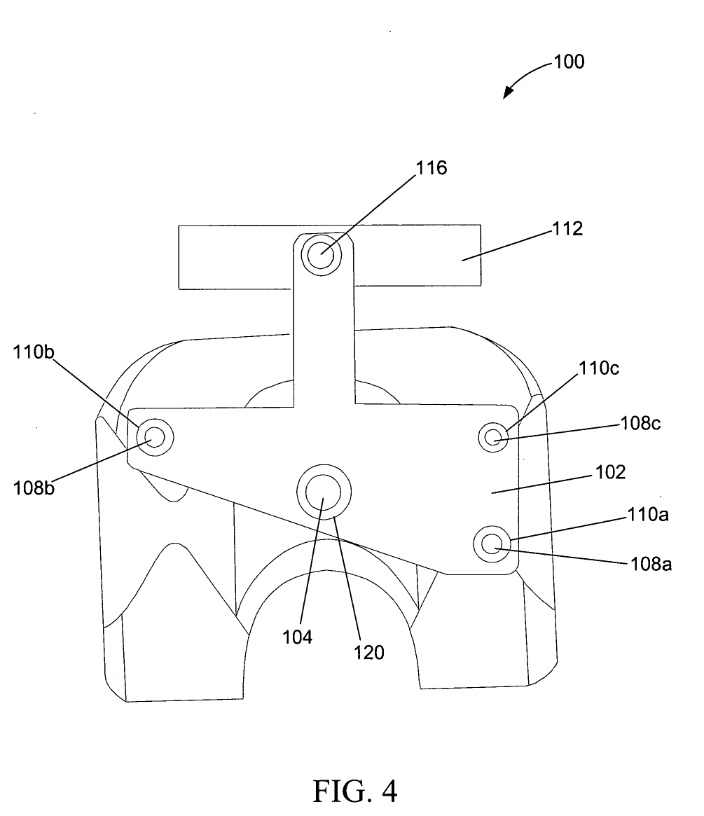

One of the embodiments of the present invention provides an adjustable cutting system (100) for performing a distal femoral cut during a TKR as illustrated in FIGS. 1-4. The adjustable cutting system (100) according to this embodiment is adjustable in one or more degrees of freedom. Various alternative embodiments may be adjustable rotationally, translationally, or both. The principles and structures of the adjustable femoral cutting system (100) illustrated can be applied to cutting systems for resection of a variety of bones, including, but not limited to, any bone resections performed during virtually any type of joint arthroplasty. The adjustable cutting system is particularly advantageous for computer-assisted surgery. For example, during computer-assisted surgery, the user provisionally locates the cutting system using conventional anatomical landmarks and then fine tunes the position using navigational feedback from the computer functionality.

Rotationally, the cutting system is adjustable in varus/valgus and flexion/extension angles. The rotational adjustment of the cutting system (100) is advantageously and accurately controlled by the adjustment of a number of adjustment members (108a-b, third adjustment member not shown) that act between a base (102) of the cutting system (100) and the surface of the bone (118). Integrating both varus/valgus and flexion/extension angular adjustment capabilities reduces the number of components as compared to conventional adjustable cutting blocks and, in one aspect, allows for reduction in size, rendering the block particularly advantageous for minimally invasive surgical applications.

The adjustable cutting system (100) comprises structures (106) for attaching the block at the bone (118), specifically, at the distal femur. For installation, the adjustable cutting system (100) may be referenced to various virtual surgical constructs, such as a mechanical axis of the femur. Prior to adjustments, the adjustable cutting system (100) is attached to or fixated at a bone (118) directly through base attachment (104) or, alternatively, by connecting it to a surgical structure, such as, but not limited to, an intramedullary rod, a post, or an adaptor. Attaching the system (100) to the bone (118) or to the surgical structure does not interfere with the adjustment capabilities, unless so desired by the surgeon. In some embodiments, after the adjustments of the block are completed, a cutting block (112) component of the system (100) is used to perform the bone resection.

In general, during a TKA the surgeon attaches the adjustable cutting system (100), adjusts the position of a cutting block (112) by adjusting adjustment members (108a-b), and performs the distal femoral cut. The system (100) comprises a base (102) and a cutting guide (112) connected by attachment (116). Upon attachment of the base (102) of the cutting system (100) to the distal end of the femur (118), the cutting guide (112) is positioned at the anterior surface of the distal femur (118) and comprises, in the lengthwise medial-lateral orientation, one or more guiding slots (114) for guiding a surgical saw in a distal femoral cut generally directed transversely to the long femoral axis.

The base also comprises an attachment (104) that attaches to the distal end of the femur (118). The attachment (104) may be a femoral anchor post or an intramedullary (IM) rod. As shown in FIGS. 1-3, in some embodiments the attachment (104) extends from the bone (118) outward and through an aperture in the base (102). The attachment (104) may include a spring (106) or other structure that biases the base (102) toward in the direction of the distal bone (118).

The attachment (104) of the base (102) to the bone (118) and the associated spring (106) will generally secure the base while still allowing the base to be rotated, translated, or otherwise positioned or adjusted. In other words, the base is held in place but has some freedom to be adjusted. Various adjustment members (108a-b) may be used to adjust the base. Adjustment member (108a-b), in FIGS. 1-4, pass through the base (102) and extend to the surface of the bone. The adjustment members (108a-b) will typically contact various portions of the distal femur (118) surface, and will typically contact the femoral condyles. The specific shape of an adjustment member (108a-b, third member not shown) and their specific interaction with the base (102) may vary in different embodiments of the system (100). Virtually any shape that allows contact with the bone (118) and any interaction with the base (102) that allows fine adjustment will work. In the embodiment of FIGS. 14, the structure of the adjustment members (108a-b) is described as a screw that interacts with threads in the base (102). Numerous alternatives are envisions such as spikes with turning knobs on the end, pins having ratchet notches, etc.

In the embodiment shown, the three adjustment members (108a-b, third member not shown) are analogous to the legs on a three legged stool. The seating surface on a three legged stool may be adjusted by altering the lengths of one or more of its legs. For example, if all three legs are lengthened or shortened in equal amounts, the seat height will raise or lower respectively. Alternatively, if less then all of the stool legs are lengthened, the plane of the seat will rotate. In the embodiment shown in FIGS. 1-4, the length of each of the adjustment members (108a-b, third member not shown) may be adjusted like the legs of the three legged stool. In some embodiments, the adjustment member (108a-b, third member not shown) configuration will form a right triangle. In other embodiments, anywhere from 1 to 10 adjustment members can be used in a virtually unlimited array of potential configuration.

Each of the adjustment members (108a-b, third member not shown) is screwed into threaded holes (110a-c) though the base (102). Adjustment member 108(a) is screwed into threaded hole (110a) and allows a flexion/extension adjustment. Adjustment member 108(b) is screwed into threaded hole (110b) and allows a varus/valgus adjustment. The third adjustment member (not shown) may go through threaded hole (110c) and provide adjustment or simply provide a fixed leg. In some alternative embodiments, the base attachment (104) will itself be an adjustment member or provide a fixed leg.

The amount of each adjustment member extending between the base (102) and the bone (118) is adjusted by turning the adjustment member. For example, as an adjustment member (108a) turns, the threads of the adjustment member (108a) engage the threads of the respective hole (110a) causing more or less of the adjustment member (108a) to extend on each side of the base (102). The three adjustment members (1 08a-b, third member not shown) are used to adjust the position of a base (102) and the attached cutting guide (112) in at least two degrees of rotational freedom, varus/valgus angle and flexion/extension.

An attachment (116) between the base (102) and the cutting guide (112) causes the position of the cutting guide (116) to change as the position of the base (102) is changed. The attachment (116) between the base (102) and the cutting block (118) may also be used to alter or otherwise adjust the resection depth of the cutting guide (118). For example, the attachment (116) could be a screw that, when turned, lengthens or shortens the distance between the base (102) and the cutting block (112).

Other attachments, connections, or adjustable arrangements between the base (102) and cutting block (118) may be used. For example, one or more ball-and-socket and/or lever controlled adjustable connections may be used as described in U.S. patent application Ser. No. 10/989,835 to McGinley et al. filed Nov. 15, 2004, incorporated herein by this reference. Such attachments may be used to add additional rotational or translational adjusting capabilities. For example, a ball-and-socket structure could permit movement of the cutting block (118) in the anterior/posterior direction or the superior/inferior direction.

The cutting guide (112) may optionally include one or more openings for inserting screws, pins, or other fixation structures to fix the guide (112) to the bone (118) after it has been properly aligned. In some embodiments, the attachment (116) of the cutting block (112) to the base (102) is capable of being disconnected. After the cutting block has been properly aligned and fixed to the bone, the surgeon may remove the base from the surgical field by disconnecting attachment (116). The surgeon then uses the one or more guiding slots (114) in the cutting guide (112) to direct the saw blade in the distal femoral cut (19).

Alternatively, the cutting block (112) does not have to be separately anchored to the bone (118) and may be held in place with respect to the bone (118) though the base (102) and its base attachment (104) and/or adjustment members (108a-b). In this case, the attachment (116) between the base (102) and the cutting block (112) will not be disengaged prior to resection. The attachment (116) may or may not be capable of disconnection.

After completing the distal femoral cut, the surgeon removes the cutting guide (13) and completes the surgery. During TKA, the surgeons often perform the distal femoral cut first when preparing the distal femur for installation of the femoral prosthetic component. Other femoral cuts follow the distal femoral cut, with the surgeon often using the distal cut's plane as a reference to establish the position of the other resection planes. In a variation on the present embodiment, adjustable cutting blocks are provided for various femoral cuts performed during TKA. For example, the adjustable cutting blocks can be provided for cuts such as, but not limited to, a transversely directed distal femoral cut, an axially directed anterior femoral cut, an axially directed posterior femoral cut, anterior and posterior chamfer femoral cuts, a trochlear recess cut, or any combination or variation of those. The cutting blocks can be combination cutting blocks suitable for performing multiple bone cuts.

In one embodiment, the surgeon uses one or more of the adjustable cutting blocks provided by certain aspects and embodiments of the present invention to perform all the cuts during a surgical procedure. For example, performing a conventional TKA femoral resection sequence of cuts, the surgeon uses an adjustable cutting block to perform a distal femoral cut. Then, using the distal plane as a reference, the surgeon employs adjustable cutting blocks to perform axial, anterior, and posterior cuts, and any other cuts, if required, not necessarily in the above order.

Adding additional adjustment capabilities, including but not limited to an additional rotational axis, is envisioned, and falls within the aspects and embodiments of the present invention. Additional angular control is advantageous, for example, for better adjustment of the cutting guide position in unicondylar knee surgery applications. Additional angular control would also be advantageous for surgical techniques, where one cutting guide facilitates all of the cuts necessary to place the total knee prosthesis. Also possible is reduction in adjustment capabilities as preferred for a particular application.

Adapting the system (100) for performing proximal tibial cuts during TKA is also envisioned. In general, the principles and concepts of the adjustable transverse cutting block (100) described herein can be applied to cutting blocks for performing various bone cuts during a range of surgical procedures, including, but not limited to, resection of bones during the joint arthroplasties.

Although suitable for bone resection during any appropriate surgical application, the adjustable transverse cutting system (100) provided herein is particularly advantageous during computer-assisted surgery. The user provisionally locates the cutting block (112) using conventional anatomical landmarks, and then fine-tunes the block's position using navigational feedback. Integrating several adjustment capabilities in the same block allows reducing the number of the block's components, as well as its size as compared to the conventional adjustable cutting blocks, thereby rendering the block according to aspects and embodiments of the present invention particularly suitable for minimally invasive surgical applications.

When the system (100) is used during TKR, the user grossly determines the position and orientation of the block, and preliminarily fixates the block at the patient's distal femur, for example, by inserting a base attachment. The user then adjusts varus/valgus and flexion/extension angles of the cutting guide using the respective adjustment members. The user rotates the appropriate knobs of one or more adjustment members, thereby adjusting a varus/valgus or flexion/extension angle of the cutting guide relative to the femur. In one embodiment, the operator first determines and adjusts the varus/valgus and flexion/extension, followed by a resection depth adjustment through an attachment between the base and the cutting guide. This order of operation, although non-limiting, can be chosen because adjusting the angular position of the cutting guide also involves translation along the long axis of the femur. The user may prefer to adjust the angular orientation of the cutting block in flexion/extension and varus/valgus, in any order, followed by the translational adjustment of the superior/inferior position, or the resection depth. After the desired position of the transverse adjustable cutting block is obtained, the user may fixate the cutting guide using appropriate fixation devices to attach the cutting guide to the femur. In one embodiment, the base is removed after the final fixation, but the base can also be left in place. Upon final fixation, the user performs the distal femoral or proximal tibial cut by using the guiding slot in the guide to direct a surgical saw.

The particular embodiments of the invention have been described for clarity, but are not limiting of the present invention. Those of skill in the art can readily determine that additional embodiments and features of the invention are within the scope of the appended claims and equivalents thereto. All publications cited herein are incorporated by reference in their entirety.

1. A system for positioning a cutting guide comprising:
an attachment member for connecting a base to bone to be resected using the cutting guide;
a base adapted to connect to the attachment member in a single connection, whereby the base is adapted to move relative to the attachment member through a plurality of positions in a predetermined range of motion in at least two degrees of rotational freedom and one degree of translational freedom; and
a plurality of adjustment members extending from the base to the bone, the adjustment members adapted to be adjusted in order to adjust the orientation of the base relative to the bone in at least two degrees of rotational freedom.

2. The system of claim 1, wherein the base includes a cutting guide.

3. The system of claim 2, wherein the cutting guide is a femoral cutting guide.

4. The system of claim 2, wherein the cutting guide is a tibial cutting guide.

5. The system of claim 1, wherein the adjustment members allow adjustment with respect to the bone in varus/valgus angle and flexion/extension angle.

6. The system of claim 1, wherein one or more of the adjustment members are screws that screw into receiving holes on the base.

7. The system of claim 1, wherein the attachment member is a femoral post.

8. The system of claim 1, wherein the attachment member is an intramedullary rod.

9. The system of claim 1, wherein the attachment member is an extramedullary rod.

10. The system of claim 1, wherein the base is connected to a cutting guide.

11. The system of claim 10, wherein the base is connected to the cutting guide by an adjustable attachment.

12. The system of claim 11, wherein the adjustable attachment is translationally adjustable.

13. The system of claim 11, wherein adjustment of the adjustable attachment is controlled by a screw.

14. The system of claim 1, wherein the base is of a size suitable for minimally invasive surgery.

15. The system of claim 1, further comprising one or more fiducials for computer-assisted surgery.

16. The system of claim 1, wherein the predetermined range of motion is continuously positionable.

17. The system of claim 1, wherein the predetermined range of motion is a fixed number of incrementally separated positions.

18. A method of arthroplasty surgery on a knee, comprising the steps of:
positioning the knee in a flexed position;
exposing a joint of the knee;
attaching a base to the bone through an attachment member, whereby the base is adapted to connect to the attachment member in a single connection and is adapted to move relative to the attachment member through a plurality of positions in a predetermined range of motion in at least two degrees of rotational freedom and one degree of translational freedom;
adjusting the position of the base by manipulating a plurality of adjustment members extending from the base to contact surfaces of the bone, whereby the adjustment members are adapted to be adjusted in order to adjust the orientation of the base relative to the bone in at least two degrees of rotational freedom; and
cutting the bone with a cutting implement directed by a cutting guide wherein the cutting guide is attached to the base.

19. The method of claim 18, further comprising adjusting the resection depth of the cutting guide by adjusting an attachment between the base and the cutting guide.

20. The method of claim 18 further comprising using one or more fiducials attached to the cutting guide to allow computer-assisted surgery.
